(19) United States
(12) Patent Application Publication
(10) Pub. No.: US 2022/0233083 A1
(43) Pub. Date: Jul. 28, 2022

(54) SPHYGMOMANOMETER, BLOOD PRESSURE MEASUREMENT METHOD, AND COMPUTER-READABLE RECORDING MEDIUM

(71) Applicant: OMRON HEALTHCARE CO., LTD., Muko-shi (JP)

(72) Inventors: Yukiya SAWANOI, Kyoto (JP); Shingo YAMASHITA, Kyoto (JP); Mika EZOE, Kyoto (JP)

(73) Assignee: OMRON HEALTHCARE CO., LTD., Muko-shi, Kyoto (JP)

(21) Appl. No.: 17/722,876

(22) Filed: Apr. 18, 2022

Related U.S. Application Data
(63) Continuation of application No. PCT/JP2020/037769, filed on Oct. 5, 2020.

(30) Foreign Application Priority Data
Oct. 24, 2019 (JP) 2019-193674

(51) Int. Cl. A61B 5/0225; A61B 5/00

(52) U.S. Cl. CPC A61B 5/0225; A61B 5/6824; A61B 2560/0475; A61B 2560/029; A61B 5/7267

(57) ABSTRACT

A sphygmomanometer has a nocturnal blood pressure measurement mode in which blood pressure measurement automatically starts according to a predetermined schedule. The sphygmomanometer includes a single operation switch for inputting an instruction to suspend or to recover the nocturnal blood pressure measurement mode, a suspension processing unit to perform a process for transitioning to a measurement suspension state in which blood pressure measurement does not start even when a set time arrives, when the single operation switch is operated at a first time, in the nocturnal blood pressure measurement mode, and a recovery processing unit to perform a process for recovering to the nocturnal blood pressure measurement mode, under condition that the single operation switch is operated at a second time or after a lapse of a predetermined time from a clock time when the single operation switch is operated at the first time, in the measurement suspension state.

### CROSS-REFERENCE TO RELATED APPLICATION

This is a continuation application of International Application No. PCT/JP2020/037769, with an International filing date of Oct. 5, 2020, which claims priority of Japanese Patent Application No. 2019-193674 filed on Oct. 24, 2019, the entire content of which is hereby incorporated by reference.

### TECHNICAL FIELD

The present invention relates to a sphygmomanometer, and more particularly to a sphygmomanometer having a nocturnal (sleep) blood pressure measurement mode. The present invention also relates to a blood pressure measurement method for measuring blood pressure using such a sphygmomanometer. The present invention also relates to a computer-readable recording medium storing a program for causing a computer to execute such a blood pressure measurement method.

### BACKGROUND ART

For example, Patent Document 1 (WO 2018/168797 A) has disclosed this type of sphygmomanometer. In the sphygmomanometer, in a nocturnal (sleep) blood pressure measurement mode, a blood pressure measurement period is specified, a measurement start time (or start clock time) and a measurement end time (or end clock time) are set, time setting is made with arbitrary time intervals (for example, one hour), and blood pressure is measured and recorded.

### SUMMARY OF THE INVENTION

Incidentally, while using the sphygmomanometer in the nocturnal blood pressure measurement mode during sleep, for example, a subject sometimes temporarily gets up to go to a bathroom. Here, the conventional sphygmomanometer may start blood pressure measurement scheduled in advance (by a built-in timer) while the subject is out of bed and moving. As is known, blood pressure measured while the subject is moving will be higher than that measured at rest. Hence, in the conventional sphygmomanometer, there is a possibility of measuring blood pressure incorrectly in the nocturnal blood pressure measurement mode.

Thus, an object of the present invention is to provide a sphygmomanometer and a blood pressure measurement method capable of preventing the start of blood pressure measurement scheduled in advance in the nocturnal blood pressure measurement mode while a subject is temporarily out of bed. Another object of the present invention is to provide a computer-readable recording medium storing a program for causing a computer to execute such a blood pressure measurement method.

In order to solve the above-mentioned problem, a sphygmomanometer of the present disclosure that performs blood pressure measurement by temporarily compressing a measurement site of a subject with a blood pressure measurement cuff,

the sphygmomanometer having a nocturnal blood pressure measurement mode in which blood pressure measurement automatically starts according to a schedule determined in advance,

the sphygmomanometer comprising:

a blood pressure measurement unit configured to automatically start blood pressure measurement according to the schedule and measure blood pressure when the blood pressure measurement cuff is in a pressurization process or a depressurization process, in the nocturnal blood pressure measurement mode;

a single operation switch for inputting an instruction to suspend the nocturnal blood pressure measurement mode or to recover to the nocturnal blood pressure measurement mode;

a suspension processing unit configured to perform a process for transitioning to a measurement suspension state in which blood pressure measurement does not start even when a clock time set in the schedule arrives, when the single operation switch is operated at a first time, in the nocturnal blood pressure measurement mode; and

a recovery processing unit configured to perform a process for recovering to the nocturnal blood pressure measurement mode, under condition that the single operation switch is operated at a second time or after a lapse of a predetermined time from a clock time when the single operation switch is operated at the first time, in the measurement suspension state.

Here, the “predetermined time” is set to five minutes, for example, assuming a time required for the subject to get up from a bed, use a bathroom, and return to the bed again. However, the present disclosure is not limited to this.

In another aspect, a blood pressure measurement method of the present disclosure for a sphygmomanometer that performs blood pressure measurement by temporarily compressing a measurement site of a subject with a blood pressure measurement cuff,

the sphygmomanometer

having a nocturnal blood pressure measurement mode in which blood pressure measurement automatically starts according to a schedule determined in advance, and

including a single operation switch for inputting an instruction to suspend the nocturnal blood pressure measurement mode or to recover to the nocturnal blood pressure measurement mode,

the blood pressure measurement method comprising:

automatically starting blood pressure measurement according to the schedule and measuring blood pressure when the blood pressure measurement cuff is in a pressurization process or a depressurization process, in the nocturnal blood pressure measurement mode;

performing a process for transitioning to a measurement suspension state in which blood pressure measurement does not start even when a clock time set in the schedule arrives, when the single operation switch is operated at a first time, in the nocturnal blood pressure measurement mode; and

performing a process for recovering to the nocturnal blood pressure measurement mode, under condition that the single operation switch is operated at a second time or after a lapse of a predetermined time from a clock time when the single operation switch is operated at the first time, in the measurement suspension state.

In yet another aspect, a computer-readable recording medium of the present disclosure is a computer-readable recording medium non-transitorily storing a program for causing a computer to execute the blood pressure measurement method.

### BRIEF DESCRIPTION OF THE DRAWINGS

The present invention will become more fully understood from the detailed description given hereinbelow and the accompanying drawings which are given by way of illustration only, and thus are not limitative of the present invention, and wherein:

FIG. 1 is a view illustrating an appearance of a wrist-type sphygmomanometer according to an embodiment of the present invention.

FIG. 2 is a diagram illustrating a block configuration of the sphygmomanometer.

FIG. 3 is a view illustrating how the sphygmomanometer is worn on a left wrist as a measurement site.

FIG. 4A is a view illustrating a sitting posture as a measurement posture.

FIG. 4B is a view illustrating a supine posture as a measurement posture.

FIG. 5 is a flowchart illustrating an operation flow of blood pressure measurement in a normal blood pressure measurement mode performed by the sphygmomanometer.

FIG. 6A is a flowchart illustrating an operation flow of blood pressure measurement in a case where, when the sphygmomanometer performs the blood pressure measurement in a nocturnal blood pressure measurement mode, a subject temporarily gets up and operates a measurement suspension switch at a first time and then operates the measurement suspension switch at a second time.

FIG. 6B is a flowchart illustrating an operation flow of a measurement suspension switch process in the operation flow of the blood pressure measurement of FIG. 6A.

FIG. 6C is a flowchart illustrating an operation flow of a blood pressure measurement process in the operation flow of the blood pressure measurement.

FIG. 6D is a diagram illustrating, with elapsed time, a relationship between operation timings of a nocturnal measurement switch and the measurement suspension switch and a measurement schedule of the nocturnal blood pressure measurement mode in the blood pressure measurement of FIG. 6A.

FIG. 6E is a diagram illustrating measurement results of the blood pressure measurement performed by the sphygmomanometer.

FIG. 7A is a flowchart illustrating an operation flow of blood pressure measurement in a case where, when the sphygmomanometer performs the blood pressure measurement in the nocturnal blood pressure measurement mode, a subject temporarily gets up and operates the measurement suspension switch at a first time, and the sphygmomanometer recovers to the nocturnal blood pressure measurement mode after a lapse of a predetermined time from a clock time when the subject operates the measurement suspension switch at a second time.

FIG. 7B is a flowchart illustrating an operation flow of a measurement suspension switch process in the operation flow of the blood pressure measurement of FIG. 7A.

FIG. 7C is a flowchart illustrating an operation flow of a measurement suspension post-recovery timer process in the operation flow of the blood pressure measurement of FIG. 7A.

FIG. 7D is a diagram illustrating, with elapsed time, a relationship between operation timings of the nocturnal measurement switch and the measurement suspension switch and a measurement schedule of the nocturnal blood pressure measurement mode in the blood pressure measurement of FIG. 7A.

FIG. 8A is a flowchart illustrating an operation flow of blood pressure measurement in a case where, when the sphygmomanometer performs the blood pressure measurement in the nocturnal blood pressure measurement mode, a subject temporarily gets up and operates the measurement suspension switch at a first time, and the sphygmomanometer recovers to the nocturnal blood pressure measurement mode after a lapse of a predetermined time from a clock time when the subject operates the measurement suspension switch at the first time.

FIG. 8B is a flowchart illustrating an operation flow of a measurement suspension switch process in the operation flow of the blood pressure measurement of FIG. 8A.

FIG. 8C is a flowchart illustrating an operation flow of a measurement suspension timer process in the operation flow of the blood pressure measurement of FIG. 8A.

FIG. 8D is a diagram illustrating, with elapsed time, a relationship between operation timings of the nocturnal measurement switch and the measurement suspension switch and a measurement schedule of the nocturnal blood pressure measurement mode in the blood pressure measurement of FIG. 8A.

### DESCRIPTION OF EMBODIMENTSMODES

Hereinafter, embodiments of the present invention will be described in detail with reference to the drawings.

### Configuration of Sphygmomanometer

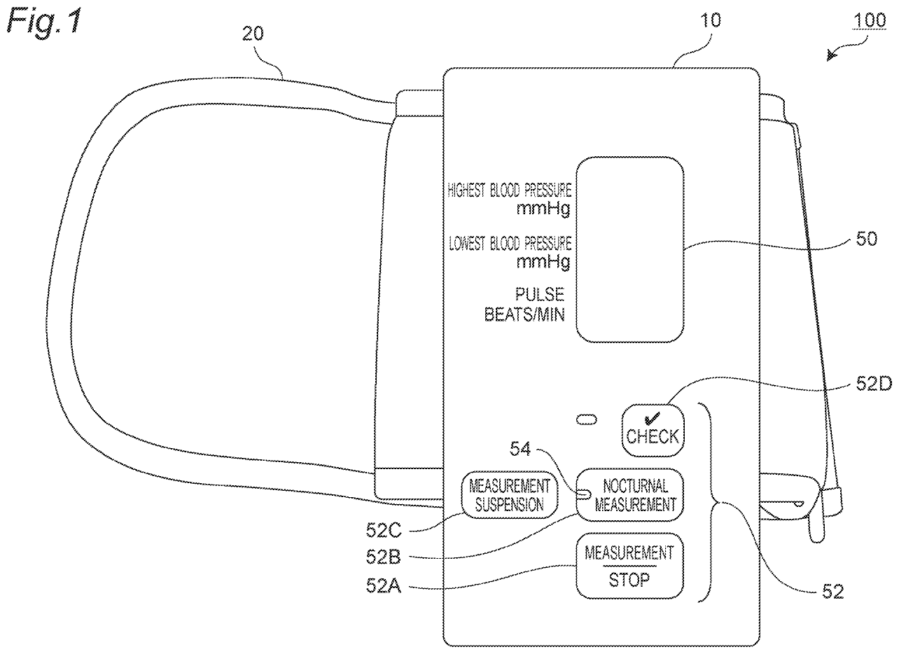

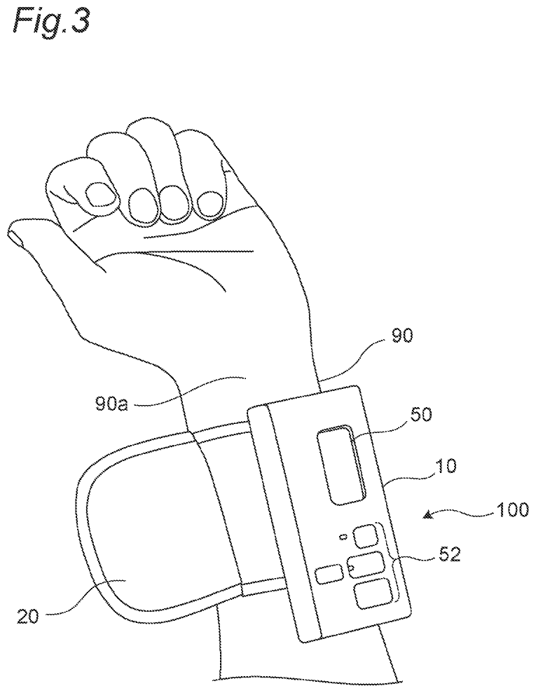

FIG. 1 illustrates an appearance of a wrist-type sphygmomanometer 100 according to an embodiment of the present invention. The sphygmomanometer 100 roughly includes a blood pressure measurement cuff 20 to be worn on a left wrist 90 as a measurement site (see FIG. 3 described later) and a main body 10 integrally attached to the cuff 20.

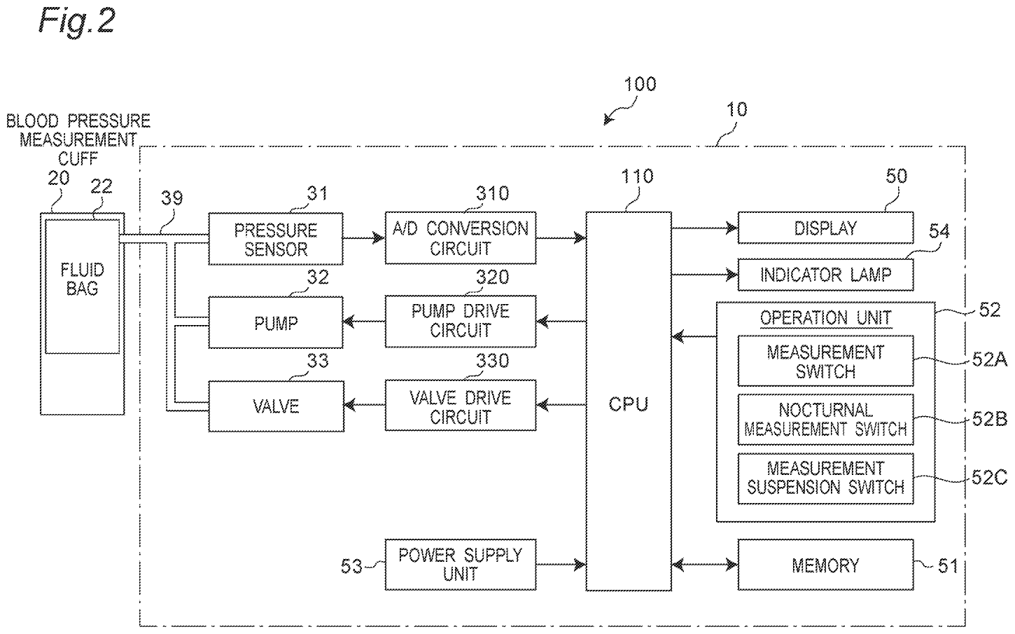

The cuff 20 is commonly used for a wrist-type sphygmomanometer, and has an elongated band shape so as to surround the left wrist 90 along a circumferential direction. The cuff 20 internally includes a fluid bag 22 (see FIG. 2) for compressing the left wrist 90. In order to always maintain the cuff 20 in an annular shape, the cuff 20 may internally include an appropriately flexible curler.

As illustrated in FIG. 3, the main body 10 is integrally attached to the band-shaped cuff 20 at a substantially central portion in a longitudinal direction. In this example, the portion where the main body 10 is attached is supposed to meet a palm-side surface (surface on a palm side of a hand) 90a of the left wrist 90 in a worn state.

The main body 10 has a flat substantially rectangular parallelepiped shape along an outer peripheral surface of the cuff 20. The main body 10 is small and thin so as not to disturb sleep of a user (in this example, referring to a subject, and the same applies hereinafter). The main body 10 has rounded corner portions (the corners are rounded).

As illustrated in FIG. 1, the main body 10 has a surface (top surface) on a side farthest from the left wrist 90 among outer surfaces. The top surface is provided with a display 50 as a display screen and an operation unit 52 for inputting an instruction from the user.

In this example, the display 50 is constituted by a liquid crystal display (LCD) and displays given information according to control signals from a central processing unit (CPU) 110 to be described later. In this example, a highest blood pressure (unit; mmHg), a lowest blood pressure (unit; mmHg), and a pulse (unit; beats/min) are displayed. The display 50 may be constituted by an organic electro luminescence (EL) display or may include light emitting diodes (LEDs).

The operation unit 52 inputs an operation signal corresponding to an instruction from the user to the CPU 110 to be described later. In this example, the operation unit 52 includes a measurement switch 52A, a nocturnal measurement switch 52B, a measurement suspension switch 52C, and a check switch 52D. The measurement switch 52A is provided for receiving a blood pressure measurement instruction from a user. The nocturnal measurement switch 52B is provided for receiving an instruction to switch between a normal blood pressure measurement mode and a nocturnal blood pressure measurement mode. The measurement suspension switch 52C is provided as a single operation switch for inputting an instruction to suspend the nocturnal blood pressure measurement mode or to recover to the nocturnal blood pressure measurement mode. The check switch 52D is provided for displaying a stored measurement result on the display unit 50. Here, the “normal blood pressure measurement mode” means a mode in which, when a blood pressure measurement instruction is input through the measurement switch 52A, blood pressure measurement is performed in response to the blood pressure measurement instruction. The “nocturnal blood pressure measurement mode” means a mode in which blood pressure measurement automatically starts according to a schedule determined in advance so that blood pressure values can be measured while the user is sleeping. The schedule determined in advance indicates a plan of measurement set at fixed clock times such as 1:00 AM, 2:00 AM, and 3:00 AM, a plan of measurement set at, for example, two-hour intervals after the nocturnal measurement switch 52B is pressed, and the like.

Specifically, in this example, each of the measurement switch 52A, the nocturnal measurement switch 52B, and the measurement suspension switch 52C is a momentary (self-restoring) switch, and is in an on state only while being pressed down and is restored to an off state when being released.

When the sphygmomanometer 100 is in the normal blood pressure measurement mode, pressing down the measurement switch 52A at a first time, which means a blood pressure measurement instruction, causes the cuff 20 to temporarily compress the measurement site (left wrist 90) for execution of blood pressure measurement by an oscillometric method. Pressing down the measurement switch 52A at a second time during the blood pressure measurement (for example, during pressurization of the cuff 20), which means an instruction to stop the blood pressure measurement, causes immediate stop of the blood pressure measurement.

When the sphygmomanometer 100 is in the normal blood pressure measurement mode, pressing down the nocturnal measurement switch 52B at a first time, which means an instruction to transition to the nocturnal blood pressure measurement mode, causes the sphygmomanometer 100 to transition from the normal blood pressure measurement mode to the nocturnal blood pressure measurement mode. In the nocturnal blood pressure measurement mode, as described above, blood pressure measurement by the oscillometric method automatically starts according to the schedule determined in advance. When the sphygmomanometer 100 is in the nocturnal blood pressure measurement mode, pressing down the nocturnal measurement switch 52B at a second time, which means an instruction to stop the nocturnal blood pressure measurement mode, causes the sphygmomanometer 100 to transition from the nocturnal blood pressure measurement mode to the normal blood pressure measurement mode.

In this example, an indicator lamp 54 is provided integrally with the nocturnal measurement switch 52B. The indicator lamp 54 is turned off while the sphygmomanometer 100 is in the normal blood pressure measurement mode. On the other hand, the indicator lamp 54 is turned on while the sphygmomanometer 100 is in the nocturnal blood pressure measurement mode. The indicator lamp 54 is temporarily turned off only while the sphygmomanometer is in a measurement suspension state to be described later. This allows the subject to check whether the sphygmomanometer 100 is in the nocturnal blood pressure measurement mode or in the measurement suspension state by viewing the indicator lamp 54.

Even when the sphygmomanometer 100 is in the nocturnal blood pressure measurement mode, the user may press the measurement switch 52A to provide an interrupting blood pressure measurement instruction separately from the schedule determined in advance. At that time, in response to the interrupting blood pressure measurement instruction, the cuff 20 temporarily compresses the measurement site (left wrist 90) for execution of blood pressure measurement by the oscillometric method.

FIG. 2 illustrates a block configuration of the sphygmomanometer 100.

As described above, the cuff 20 includes the fluid bag 22 for compressing the left wrist 90 as the measurement site. The fluid bag 22 is connected to the main body 10 by an air pipe 39 for a fluid to be able to flow.

The main body 10 is equipped with, in addition to the display 50 and the operation unit 52 described above, the CPU 110 as a control unit, a memory 51 as a storage unit, a power supply unit 53, a pressure sensor 31, a pump 32, and a valve 33. The main body 10 is further equipped with an A/D conversion circuit 310 that converts output of the pressure sensor 31 from an analog signal to a digital signal, a pump drive circuit 320 that drives the pump 32, and a valve drive circuit 330 that drives the valve 33. The pressure sensor 31, the pump 32, and the valve 33 are connected to the fluid bag 22 through the air pipe 39 in common for a fluid to be able to flow.

The memory 51 stores a program for controlling the sphygmomanometer 100, data used for controlling the sphygmomanometer 100, setting data for setting various functions of the sphygmomanometer 100, measurement result data of blood pressure values, and the like. The memory 51 is also used as a work memory when the program is executed or the like. Particularly, in this example, the memory 51 stores an algorithm for blood pressure calculation by the oscillometric method.

The CPU 110 shown in FIG. 2 controls the entire operation of the sphygmomanometer 100. Specifically, the CPU 110 works as a blood pressure measurement unit, and controls driving of the pump 32 and the valve 33 in response to operation signals from the operation unit 52 according to the program for controlling the sphygmomanometer 100 stored in the memory 51. The CPU 110 works as the blood pressure measurement unit, and in the nocturnal blood pressure measurement mode, automatically starts blood pressure measurement according to the schedule and measures blood pressure using the algorithm for blood pressure calculation by the oscillometric method when the blood pressure measurement cuff is in a pressurization process or a depressurization process. The CPU 110 also works as a suspension processing unit, and performs a process for transitioning to the measurement suspension state in which blood pressure measurement does not start even when a clock time set in the schedule arrives, when the measurement suspension switch 52C is turned on at a first time. The CPU 110 also works as a recovery processing unit, and in the measurement suspension state, performs a process for recovering to the nocturnal blood pressure measurement mode, under condition that the measurement suspension switch 52C is turned on at a second time or after a lapse of a predetermined time from a clock time when the measurement suspension switch 52C is turned on at the first time. These processes will be described in detail later.

In this example, the power supply unit 53 is constituted by a secondary battery, and supplies power to each of the CPU 110, the pressure sensor 31, the pump 32, the valve 33, the display 50, the memory 51, the A/D conversion circuit 310, the pump drive circuit 320, and the valve drive circuit 330.

The pump 32 supplies air as the fluid into the fluid bag 22 through the air pipe 39 to increase pressure (cuff pressure) in the fluid bag 22 included in the cuff 20. The valve 33 is opened and closed to control the cuff pressure by releasing or trapping the air in the fluid bag 22 through the air pipe 39. The pump drive circuit 320 drives the pump 32 based on a control signal from the CPU 110. The valve drive circuit 330 opens and closes the valve 33 based on a control signal from the CPU 110.

The pressure sensor 31 and the A/D conversion circuit 310 work as a pressure detection unit that detects the pressure of the cuff. In this example, the pressure sensor 31 is a piezoresistive pressure sensor, and outputs the pressure (cuff pressure) in the fluid bag 22 included in the cuff 20 as an electrical resistance due to a piezoresistive effect through the air pipe 39. The A/D conversion circuit 310 converts the output (electrical resistance) of the pressure sensor 31 from an analog signal to a digital signal and outputs the converted signal to the CPU 110. In this example, the A/D conversion circuit 310 works as an oscillation circuit that oscillates at a frequency corresponding to the electrical resistance from the pressure sensor 31. The CPU 110 acquires a signal indicating the cuff pressure based on the oscillation frequency.

### Blood Pressure Calculation Method

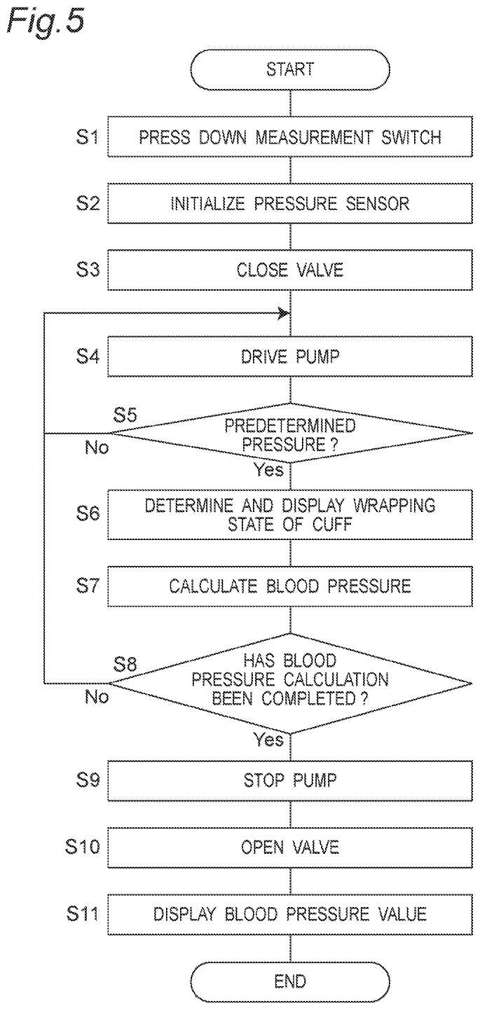

FIG. 5 illustrates an operation flow when a user uses the sphygmomanometer 100 to perform blood pressure measurement in the normal blood pressure measurement mode. In this example, pressing the measurement switch 52A continuously for, for example, 3 seconds or more in a power-off state causes the sphygmomanometer to be powered on in the normal blood pressure measurement mode by default.

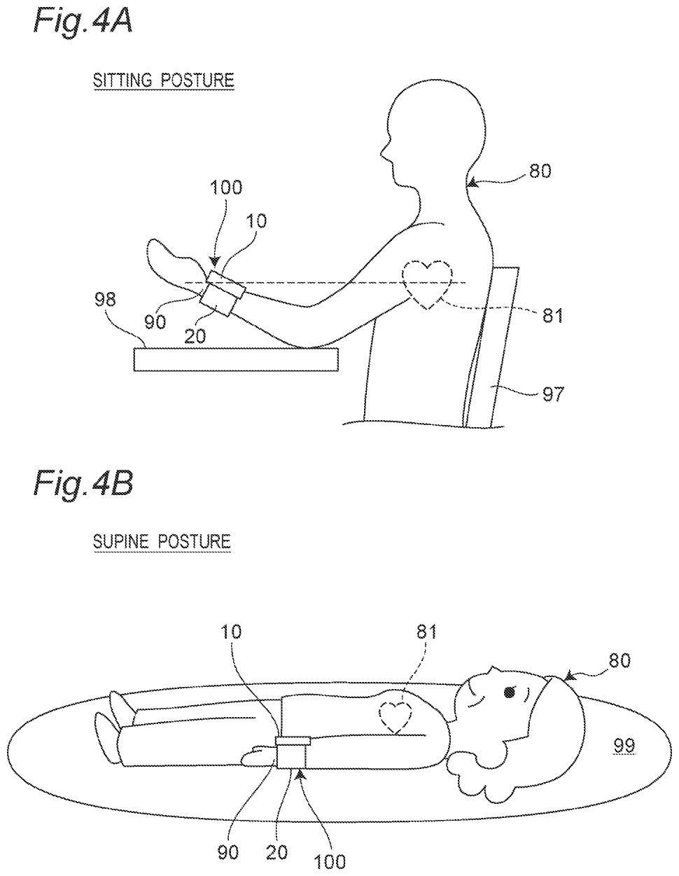

As illustrated in FIG. 4A, it is assumed that a user 80 wearing the sphygmomanometer 100 on the left wrist 90 is in a sitting posture.

Here, as illustrated in FIG. 4A, the “sitting posture” means a posture in which the user 80 wearing the sphygmomanometer 100 on the left wrist 90 sits on a chair 97 or the like, and holds the left wrist 90 (and the sphygmomanometer 100) at a height level of a heart 81 by raising the left wrist 90 obliquely (hand up, elbow down) in front of a trunk with a left elbow on a table 98. On the other hand, as illustrated in FIG. 4B, a “supine posture” means a posture in which the user 80 wearing the sphygmomanometer 100 on the left wrist 90 lies on his/her back on a horizontal floor 99 or the like with the left elbow extended along the trunk.

As shown in step S1 in FIG. 5, when the user presses down the measurement switch 52A provided on the main body 10 to input a blood pressure measurement instruction, the CPU 110 initializes the pressure sensor 31 (step S2). Specifically, the CPU 110 initializes a processing memory area and performs 0 mmHg adjustment (sets an atmospheric pressure to 0 mmHg) on the pressure sensor 31 with the pump 32 off (stopped) and the valve 33 open.

Next, the CPU 110 closes the valve 33 via the valve drive circuit 330 (step S3), and then turns on (activates) the pump 32 via the pump drive circuit 320 to start pressurization of the cuff 20 (fluid bag 22) (step S4). At this time, the CPU 110 controls an increase rate of a cuff pressure PC, which is the pressure in the fluid bag 22, based on the output of the pressure sensor 31 while supplying air from the pump 32 to the fluid bag 22 through the air pipe 39.

Next, in step S5 in FIG. 5, the CPU 110 works as a pressure measurement unit, and determines whether a predetermined pressure is reached. When the predetermined pressure is reached (Yes in step S5), a wrapping state of the cuff 20 is determined and displayed (step S6). The wrapping state can be determined by a publicly known technique as disclosed in, for example, the specification of Japanese Patent No. 5408142. On the other hand, when the predetermined pressure is not reached (No in step S5), the pressurization of the cuff 20 is continued.

Next, in step S7 in FIG. 5, calculation of blood pressure values (highest blood pressure (systolic blood pressure) and lowest blood pressure (diastolic blood pressure)) is attempted using the algorithm for blood pressure calculation stored in the memory 51 based on currently acquired pulse wave signals (fluctuation components due to the pulse wave included in the output of the pressure sensor 31).

When the blood pressure values cannot be calculated yet at this point due to lack of data (No in step S8), the processing of steps S4 to S8 is repeated until the cuff pressure PC reaches an upper limit pressure (for example, set in advance to 300 mmHg for safety).

When the blood pressure values can be calculated in this manner (Yes in step S8), the CPU 110 turns off the pump 32 (step S9) and opens the valve 33 (step S10) to control the release of the air in the cuff 20 (fluid bag 22).

Thereafter, the CPU 110 displays the calculated blood pressure values on the display 50 (step S11), and controls storing of the blood pressure values in the memory 51.

### First Embodiment

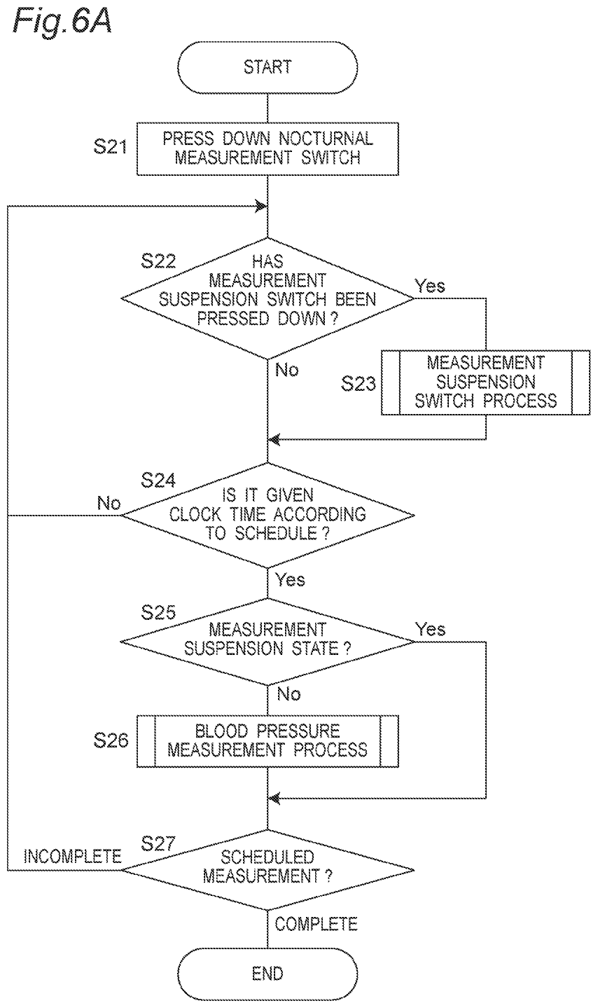

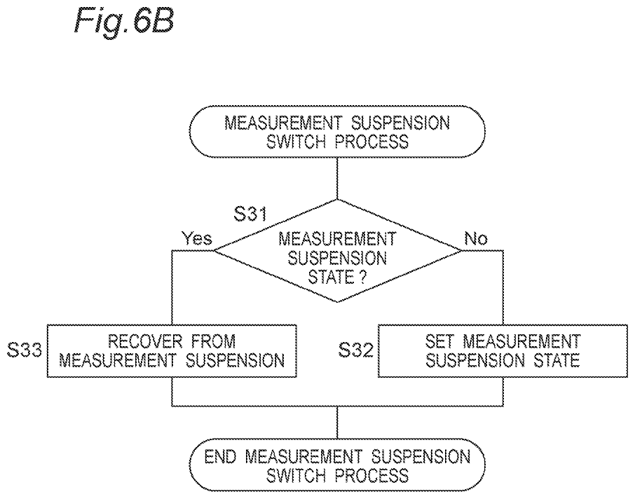

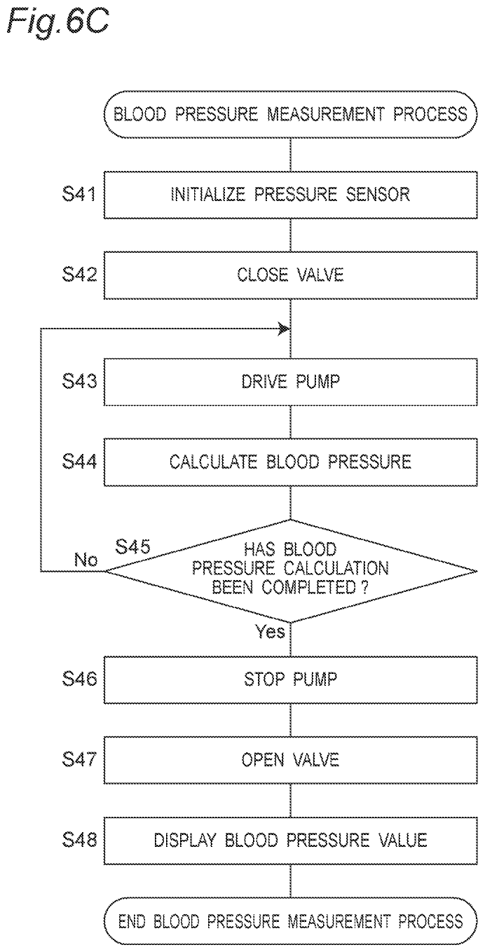

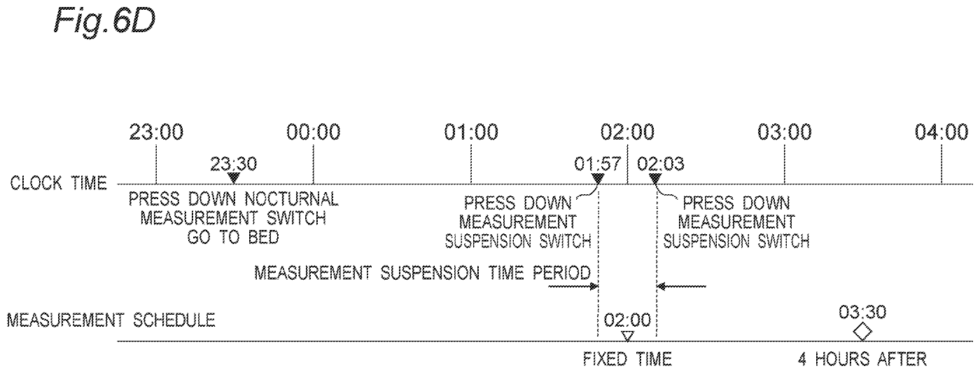

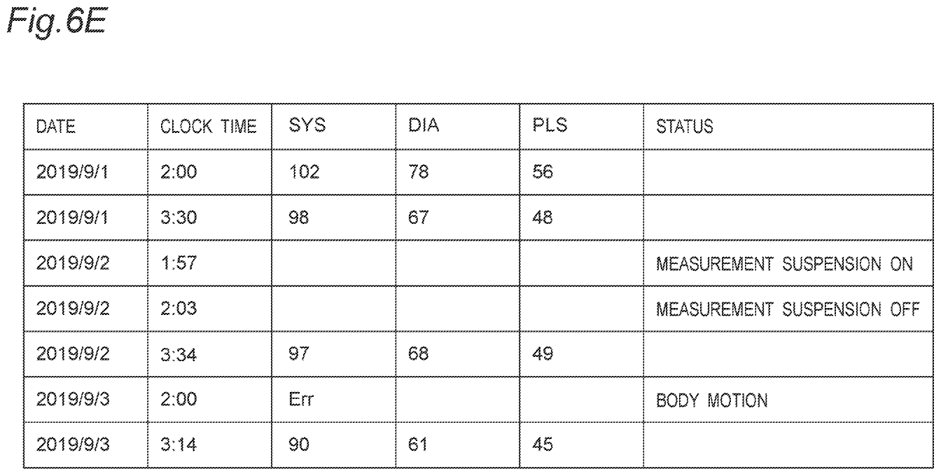

FIG. 6A illustrates an operation flow of blood pressure measurement in a case where, when using the sphygmomanometer 100 to perform the blood pressure measurement in the nocturnal blood pressure measurement mode, a user temporarily gets up, for example, to go to a bathroom and turns on the measurement suspension switch 52C at a first time and then turns on the measurement suspension switch 52C at a second time. Here, it is assumed that the user 80 wearing the sphygmomanometer 100 on the left wrist 90 is in the supine posture as illustrated in FIG. 4B.

As shown in step S21 in FIG. 6A, when the user presses down the nocturnal measurement switch 52B provided on the main body 10, the sphygmomanometer 100 transitions from the normal blood pressure measurement mode to the nocturnal blood pressure measurement mode. At this time, the indicator lamp 54 (see FIG. 1) is turned on in the sphygmomanometer 100. This allows the user to check that the sphygmomanometer 100 is in the nocturnal blood pressure measurement mode by viewing the indicator lamp 54. In this example, as illustrated in FIG. 6D, it is assumed that the nocturnal measurement switch 52B is pressed at 11:30 PM for transition to the nocturnal blood pressure measurement mode. It is also assumed that there is determined a schedule in which, for example, measurement is set at a fixed clock time of 2:00 AM and at 3:30 AM, that is 4 hours after a clock time when the nocturnal measurement switch 52B is pressed (note that, in FIG. 6D and FIGS. 7D and 8D described later, time is shown in 24-hour notation, such as 23:30).

As shown in step S22 in FIG. 6A, the CPU 110 determines whether the user has pressed down the measurement suspension switch 52C provided on the main body 10. When the user has pressed down the measurement suspension switch 52C provided on the main body 10 (Yes in step S22), the CPU 110 works as the suspension processing unit for transition to a measurement suspension switch process (step S23). At this time, the indicator lamp 54 is turned off in the sphygmomanometer 100. This allows the user to check that the sphygmomanometer 100 is in the measurement suspension state by viewing the indicator lamp 54. In this example, as illustrated in FIG. 6D, it is assumed that the user presses down the measurement suspension switch 52C at 1:57 AM.

As shown in step S31 in FIG. 6B, in the measurement suspension switch process, the CPU 110 determines whether the sphygmomanometer 100 is in the measurement suspension state. When the sphygmomanometer 100 is not in the measurement suspension state (No in step S31), the CPU 110 works as the suspension processing unit and sets the measurement suspension state (step S32). In this example, the CPU 110 sets a suspension flag in the memory 51. Thereafter, the measurement suspension switch process is ended, and the processing returns to step S24 in FIG. 6A.

As shown in step S24 in FIG. 6A, the CPU determines whether it is a measurement clock time according to the schedule of the nocturnal blood pressure measurement mode. When it is not a measurement clock time according to the schedule (No in step S24), the processing returns to step S22, and the CPU determines whether the user has pressed down the measurement suspension switch 52C. When the user has not pressed down the measurement suspension switch 52C (No in step S22), the sphygmomanometer 100 waits for a measurement clock time according to the schedule.

As shown in step S24 in FIG. 6A, the CPU 110 determines whether it is a measurement clock time according to the schedule of the nocturnal blood pressure measurement mode. When it is a measurement clock time according to the schedule (Yes in step S24), the CPU 110 subsequently determines whether the measurement suspension state has been set. When the measurement suspension state has been set (Yes in step S25), the sphygmomanometer 100 cancels a blood pressure measurement process (step S26). In this example, as illustrated in FIG. 6D, the measurement set at 2:00 AM in the schedule is canceled.

As shown in step S27 in FIG. 6A, the CPU 110 determines whether the measurement set in the schedule of the nocturnal blood pressure measurement mode has been completed. When the given measurement has not been completed (incomplete in step S27), the processing returns to step S22.

As shown in step S22 in FIG. 6A, during standby, the CPU 110 determines whether the user has pressed down the measurement suspension switch 52C provided on the main body 10. When the user has pressed down the measurement suspension switch 52C at a second time, for example, after going to the bathroom (Yes in step S22), the sphygmomanometer 100 transitions to the measurement suspension switch process (step S23). In this example, as illustrated in FIG. 6D, it is assumed that the user presses down the measurement suspension switch 52C at 2:03 AM.

As shown in step S31 in FIG. 6B, in the measurement suspension switch process, the CPU 110 determines whether the sphygmomanometer 100 is in the measurement suspension state. When the sphygmomanometer 100 is in the measurement suspension state (Yes in step S31), the CPU 110 works as the recovery processing unit and resets the measurement suspension state for recovery from the measurement suspension state (step S33). Thereafter, the measurement suspension switch process is ended, and the sphygmomanometer 100 recovers to the nocturnal blood pressure measurement mode. In this example, the sphygmomanometer 100 turns on the indicator lamp 54 to indicate that the measurement suspension switch 52C has been turned on at the second time. This allows the user to easily check the recovery to the nocturnal blood pressure measurement mode.

As shown in step S24 in FIG. 6A, the CPU 110 determines whether it is a measurement clock time according to the schedule of the nocturnal blood pressure measurement mode. When it is a measurement clock time according to the schedule (Yes in step S24), the CPU 110 subsequently determines whether the measurement suspension state has been set. When the measurement suspension state has been reset (No in step S25), the sphygmomanometer 100 proceeds to the blood pressure measurement process (step S26). In this example, as illustrated in FIG. 6D, the measurement set at 3:30 AM in the schedule is performed.

In the blood pressure measurement process shown in step S26 in FIG. 6A, the CPU 110 works as the blood pressure measurement unit and measures blood pressure. As illustrated in FIG. 6C, the blood pressure measurement process is performed according to steps similar to steps S2 to S11 excluding steps S5 and S6 in FIG. 5 described above. As shown in step S27 in FIG. 6A, the CPU 110 subsequently determines whether the measurement set in the schedule of the nocturnal blood pressure measurement mode has been completed. When all the given measurement has been completed (complete in step S27), the nocturnal blood pressure measurement mode of the sphygmomanometer 100 is ended. At this time, the indicator lamp 54 is turned off.

Therefore, according to the sphygmomanometer 100, it is possible to prevent the start of blood pressure measurement scheduled in advance in the nocturnal blood pressure measurement mode while the subject is temporarily out of bed.

In this example, as illustrated in FIG. 6E, the sphygmomanometer 100 stores measurement results in the memory 51 as follows. At 2:00 AM on Sep. 1, 2019, the highest blood pressure (systolic blood pressure (SYS))=102 mmHg, the lowest blood pressure (diastolic blood pressure (DIA))=78 mmHg, and the pulse (PLS)=56 times/min were stored. At 3:30 AM on the same day, the systolic blood pressure (SYS)=98 mmHg, the diastolic blood pressure (DIA)=68 mmHg, and the pulse (PLS)=48 times/min were stored. Next, at 1:57 AM on the next day, September 2, the user pressed down the measurement suspension switch 52C during measurement in the nocturnal blood pressure measurement mode. As a result, the measurement set in advance at 2:00 AM in the schedule was canceled, and the blood pressure measurement was not executed. At 2:03 AM, the user pressed down the measurement suspension switch 52C, and thus the sphygmomanometer recovered to be in the nocturnal blood pressure measurement mode thereafter. Then, at 3:34 AM, that is 4 hours after a clock time when the nocturnal measurement switch 52B was pressed, the highest blood pressure (systolic blood pressure (SYS))=97 mmHg, the lowest blood pressure (diastolic blood pressure (DIA))=68 mmHg, and the pulse (PLS)=49 times/min were stored. Next, at 2:00 AM on the next day, September 3, no measurement result was stored because the sphygmomanometer 100 had an operation error due to body motion of the subject. At 3:14 AM, that is 4 hours after a clock time when the nocturnal measurement switch 52B was pressed, on the same day, September 3, the highest blood pressure (systolic blood pressure (SYS))=90 mmHg, the lowest blood pressure (diastolic blood pressure (DIA))=61 mmHg, and the pulse (PLS)=45 times/min were stored.

### Second Embodiment

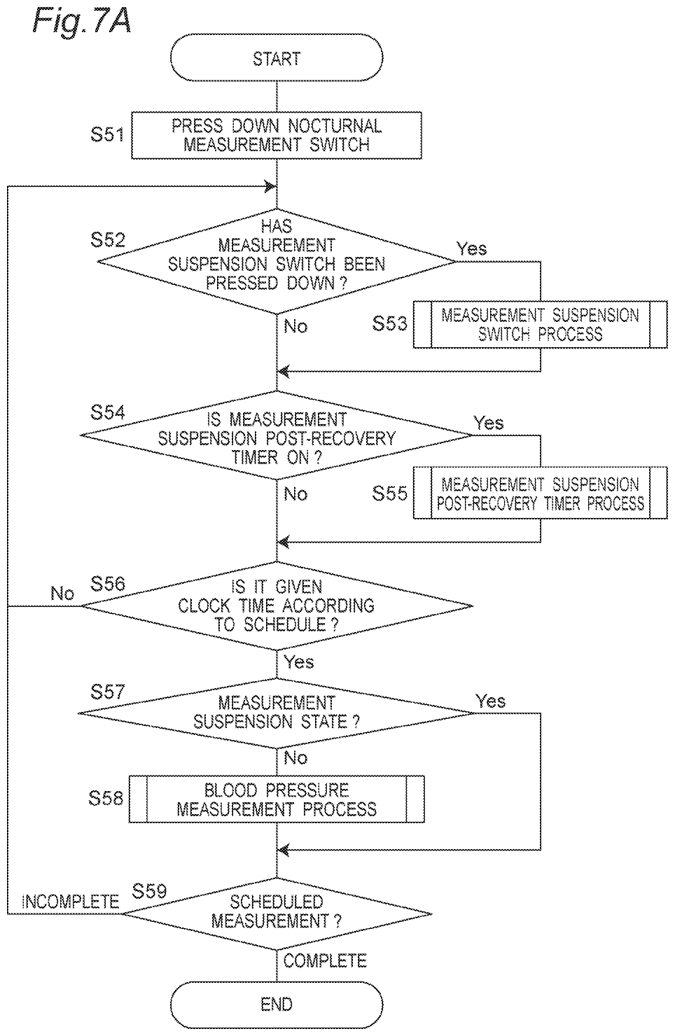

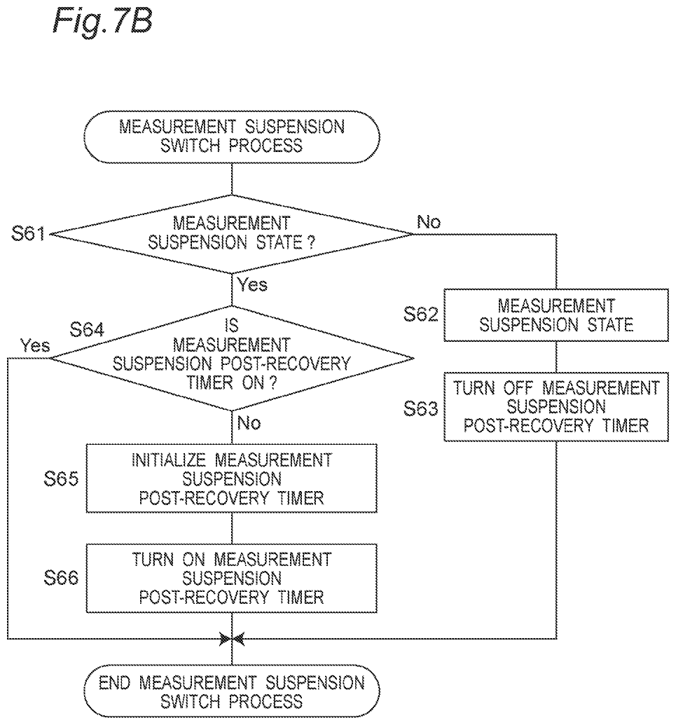

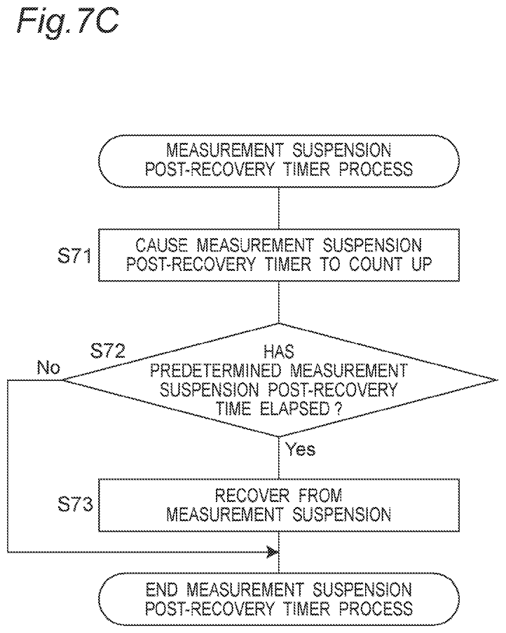

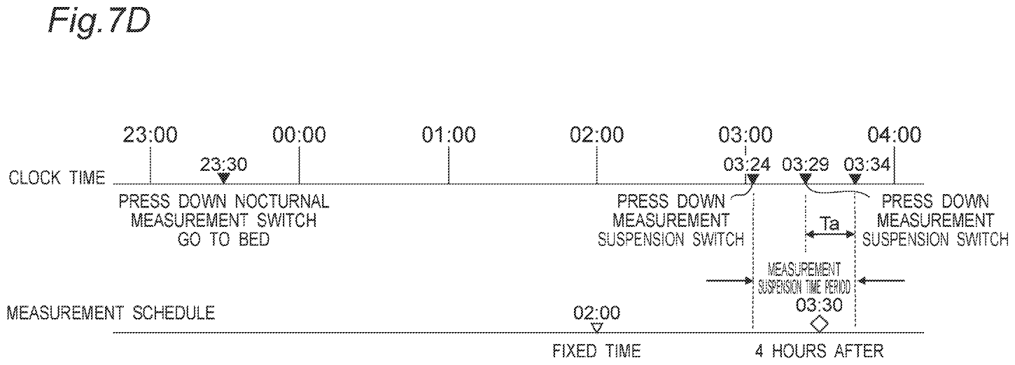

FIG. 7A illustrates an operation flow of blood pressure measurement in a case where, when using the sphygmomanometer 100 to perform the blood pressure measurement in the nocturnal blood pressure measurement mode, a user temporarily gets up and turns on the measurement suspension switch 52C at a first time, and the sphygmomanometer 100 recovers to the nocturnal blood pressure measurement mode after a lapse of a predetermined time from a clock time when the measurement suspension switch 52C is turned on at a second time. In this example, as illustrated in FIG. 7D, it is assumed that the nocturnal measurement switch 52B is pressed at 11:30 PM for transition to the nocturnal blood pressure measurement mode. It is also assumed that there is determined a schedule in which, for example, measurement is set at a fixed clock time of 2:00 AM and at 3:30 AM, that is 4 hours after a clock time when the nocturnal measurement switch 52B is pressed. In this example, the user presses down the measurement suspension switch 52C at the first time at 3:24 AM, and then presses down the measurement suspension switch 52C at the second time at 3:29 AM. Subsequently, the sphygmomanometer 100 automatically recovers to the nocturnal blood pressure measurement mode at 3:34 AM, that is after a lapse of a predetermined time Ta (in this example, five minutes).

As shown in step S51 in FIG. 7A, when the user presses down the nocturnal measurement switch 52B provided on the main body 10, the sphygmomanometer 100 transitions from the normal blood pressure measurement mode to the nocturnal blood pressure measurement mode. At this time, the indicator lamp 54 is turned on in the sphygmomanometer 100.

As shown in step S52 in FIG. 7A, the CPU 110 determines whether the user has pressed down the measurement suspension switch 52C provided on the main body 10. When the user has pressed down the measurement suspension switch 52C provided on the main body 10 (Yes in step S52), the sphygmomanometer 100 transitions to a measurement suspension switch process (step S53).

As shown in step S61 in FIG. 7B, in the measurement suspension switch process, the CPU 110 determines whether the sphygmomanometer 100 is in the measurement suspension state. When the sphygmomanometer 100 is not in the measurement suspension state (No in step S61), the CPU 110 works as the suspension processing unit and sets the measurement suspension state (step S62). At this time, the indicator lamp 54 is turned off. Subsequently, the CPU 110 turns off a measurement suspension post-recovery timer (step S63). Thereafter, the measurement suspension switch process is ended, and the processing returns to step S54 in FIG. 7A.

As shown in step S54 in FIG. 7A, the CPU 110 determines whether the measurement suspension post-recovery timer of the sphygmomanometer 100 is on. When the measurement suspension post-recovery timer is not on (No in step S54), the CPU determines whether it is a measurement clock time according to the schedule of the nocturnal blood pressure measurement mode (step S56). When it is not a measurement clock time according to the schedule (No in step S56), the processing returns to step S52, and it is determined whether the user has pressed down the measurement suspension switch 52C. When the user has not pressed down the measurement suspension switch 52C (No in step S52), the sphygmomanometer 100 waits for a measurement clock time according to the schedule.

As shown in step S52 in FIG. 7A, during standby, the CPU 110 determines whether the user has pressed down the measurement suspension switch 52C provided on the main body 10. When the user has presses down the measurement suspension switch 52C provided on the main body 10 at the second time (Yes in step S52), the sphygmomanometer 100 transitions to the measurement suspension switch process (step S53). In this example, as illustrated in FIG. 7D, it is assumed that the user presses down the measurement suspension switch 52C at the second time at 3:29 AM.

As shown in step S61 in FIG. 7B, in the measurement suspension switch process, the CPU 110 determines whether the sphygmomanometer 100 is in the measurement suspension state. When the sphygmomanometer 100 is in the measurement suspension state (Yes in step S61), the CPU determines whether the measurement suspension post-recovery timer is on. When the measurement suspension post-recovery timer is not on (No in step S64), the CPU 110 works as the recovery processing unit and initializes the measurement suspension post-recovery timer (step S65). Subsequently, the CPU 110 works as the recovery processing unit and turns on the measurement suspension post-recovery timer (step S66). Thereafter, the measurement suspension switch process is ended, and the processing returns to step S54 in FIG. 7A.

As shown in step S54 in FIG. 7A, the CPU 110 determines whether the measurement suspension post-recovery timer of the sphygmomanometer 100 is on. When the measurement suspension post-recovery timer is on (Yes in step S54), the sphygmomanometer 100 transitions to a measurement suspension post-recovery timer process (step S55).

As shown in step S71 in FIG. 7C, in the measurement suspension post-recovery timer process, the CPU 110 works as the recovery processing unit and causes the measurement suspension post-recovery timer to count up. Subsequently, the CPU 110 works as the recovery processing unit and determines whether the predetermined measurement suspension post-recovery time Ta has elapsed (step S72). When the predetermined measurement suspension recovery time Ta has not elapsed (No in step S72), the measurement suspension post-recovery timer process is ended, and the processing returns to step S56 in FIG. 7A. In this example, the predetermined time Ta is set to five minutes, for example, assuming a time required for the user to enter a resting state after pressing the measurement suspension switch 52C at the second time. However, the present disclosure is not limited to this.

In a case where the predetermined measurement suspension recovery time Ta has elapsed, as shown in step S72 in FIG. 7C, the CPU 110 works as the recovery processing unit and determines whether the predetermined measurement suspension post-recovery time Ta has elapsed. When determining that the predetermined measurement suspension recovery time Ta has elapsed (Yes in step S72), the CPU 110 resets the measurement suspension state for recovery from the measurement suspension state (step S73). Thereafter, the measurement suspension post-recovery timer process is ended, and the processing returns to step S56 in FIG. 7A.

As shown in step S56 in FIG. 7A, the CPU 110 determines whether it is a measurement clock time according to the schedule of the nocturnal blood pressure measurement mode. When it is a measurement clock time according to the schedule (Yes in step S56), the CPU 110 subsequently determines whether the measurement suspension state has been set. When the measurement suspension state has been set (Yes in step S57), the sphygmomanometer 100 cancels a blood pressure measurement process (step S58). In this example, as illustrated in FIG. 7D, the measurement set at 3:30 AM is canceled.

In a case where the measurement suspension state has been reset, as shown in step S57 in FIG. 7A, the CPU 110 determines whether the measurement suspension state has been set. When it is determined that the measurement suspension state has been reset (No in step S57), the sphygmomanometer 100 proceeds to the blood pressure measurement process (step S58). In the blood pressure measurement process shown in step S58, the CPU 110 works as the blood pressure measurement unit and measures blood pressure. As illustrated in FIG. 6C, the blood pressure measurement process is performed according to steps similar to steps S2 to S11 excluding steps S5 and S6 in FIG. 5 described above.

As shown in step S59 in FIG. 7A, the CPU 110 determines whether the measurement set in the schedule of the nocturnal blood pressure measurement mode has been completed. When all the given measurement has been completed (Yes in step S59), the nocturnal blood pressure measurement mode of the sphygmomanometer 100 is ended.

Thus, in the sphygmomanometer 100, the process for recovering to the nocturnal blood pressure measurement mode is performed after a lapse of the predetermined time Ta from the clock time when the measurement suspension switch 52C is turned on at the second time. As a result, it is possible to continue the nocturnal blood pressure measurement mode after waiting for the user to enter the resting state.

### Third Embodiment

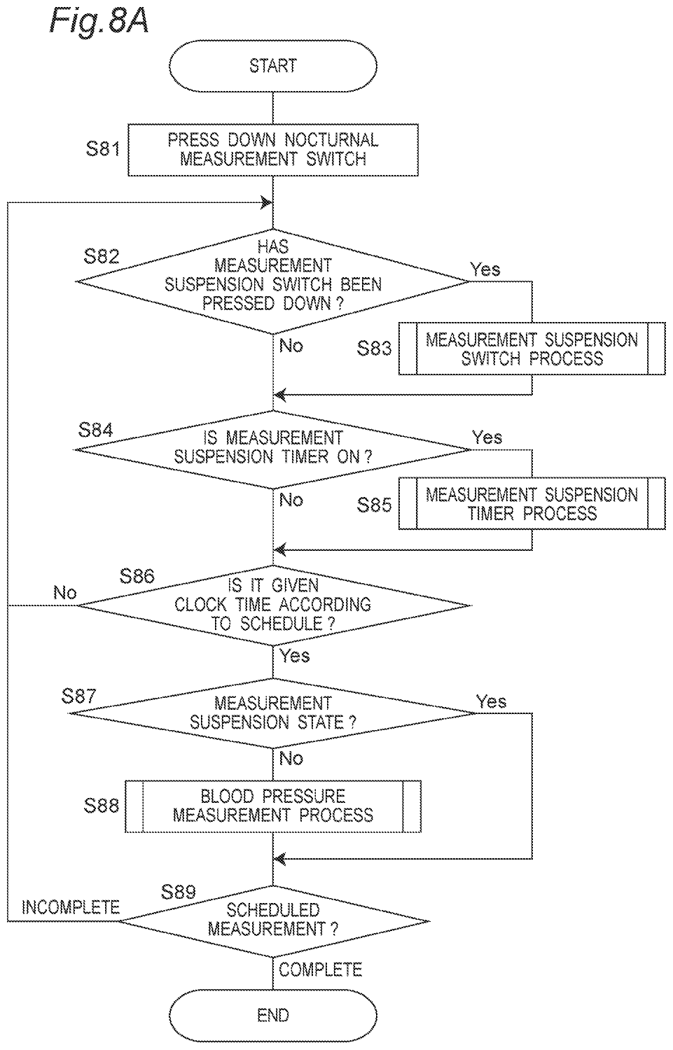

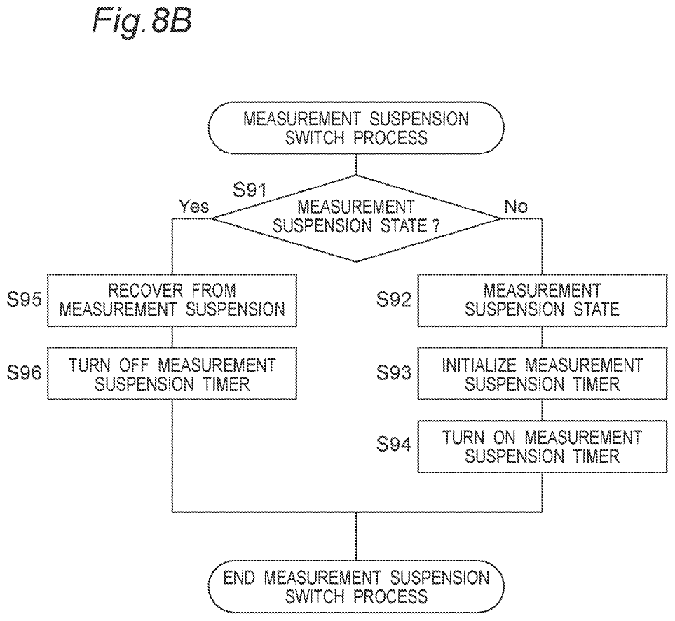

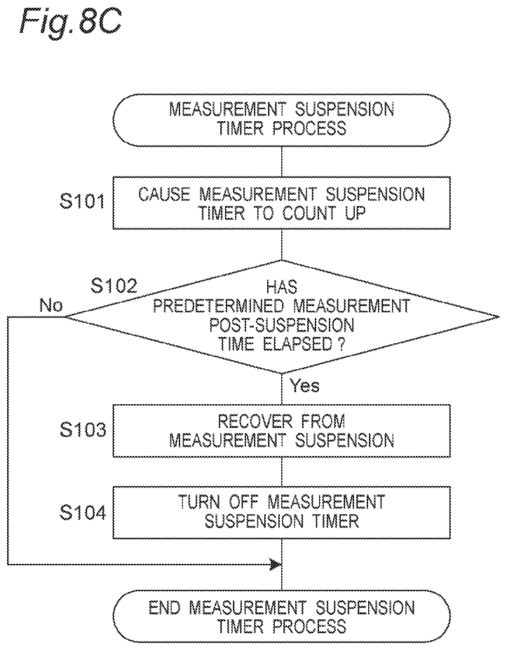

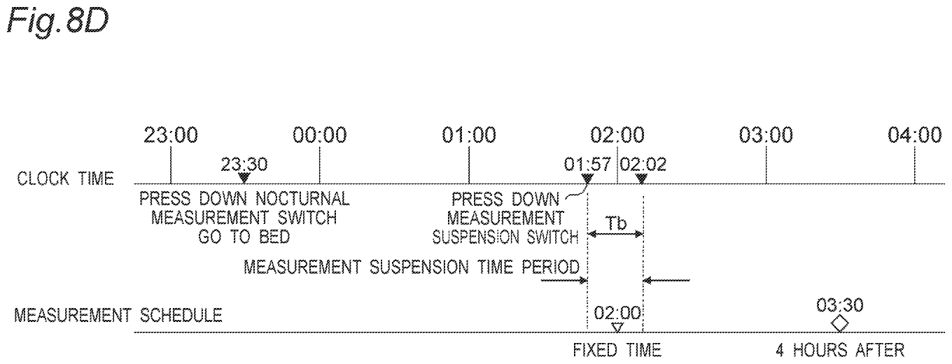

FIG. 8A illustrates an operation flow of blood pressure measurement in a case where, when using the sphygmomanometer 100 to perform the blood pressure measurement in the nocturnal blood pressure measurement mode, a user temporarily gets up and turns on the operation switch at a first time, and the sphygmomanometer 100 recovers to the nocturnal blood pressure measurement mode after a lapse of a predetermined time from a clock time when the single operation switch is turned on at the first time. In this example, as illustrated in FIG. 8D, it is assumed that the nocturnal measurement switch 52B is pressed at 11:30 PM for transition to the nocturnal blood pressure measurement mode. It is also assumed that there is determined a schedule in which, for example, measurement is set at a fixed clock time of 2:00 AM and at 3:30 AM, that is 4 hours after a clock time when the nocturnal measurement switch 52B is pressed. In this example, as illustrated in FIG. 8D, it is assumed that the user presses down the measurement suspension switch 52C at the first time at 1:57 AM. Then, the sphygmomanometer 100 automatically recovers to the nocturnal blood pressure measurement mode at 2:02 AM, that is after a lapse of a predetermined time Tb (in this example, five minutes).

As shown in step S81 in FIG. 8A, when the user presses down the nocturnal measurement switch 52B provided on the main body 10, the sphygmomanometer 100 transitions from the normal blood pressure measurement mode to the nocturnal blood pressure measurement mode. At this time, the indicator lamp 54 is turned on in the sphygmomanometer 100.

As shown in step S82 in FIG. 8A, the CPU 110 determines whether the user has pressed down the measurement suspension switch 52C provided on the main body 10. When the user has pressed down the measurement suspension switch 52C provided on the main body 10 (Yes in step S82), the sphygmomanometer 100 transitions to a measurement suspension switch process (step S83). In this example, it is assumed that the user presses down the measurement suspension switch 52C at 1:57 AM.

As shown in step S91 in FIG. 8B, in the measurement suspension switch process, the CPU 110 determines whether the sphygmomanometer 100 is in the measurement suspension state. When the sphygmomanometer 100 is not in the measurement suspension state (No in step S91), the CPU 110 works as the suspension processing unit and sets the measurement suspension state (step S92). At this time, the indicator lamp 54 is turned off. Subsequently, the CPU 110 initializes a measurement suspension timer (step S93). Subsequently, the CPU 110 turns on the measurement suspension timer (step S94). Thereafter, the measurement suspension switch process is ended, and the processing returns to step S84 in FIG. 8A.

As shown in step S84 in FIG. 8A, the CPU 110 determines whether the measurement suspension timer of the sphygmomanometer 100 is on. When the measurement suspension timer is on (Yes in step S84), the sphygmomanometer 100 transitions to a measurement suspension timer process (step S85).

As shown in step S101 in FIG. 8C, in the measurement suspension timer process, the CPU 110 works as the recovery processing unit and causes the measurement suspension timer to count up. Subsequently, the CPU 110 determines whether the predetermined measurement post-suspension time Tb has elapsed (step S102). When the predetermined measurement post-suspension time Tb has not elapsed (No in step S102), the measurement suspension timer process is ended, and the processing returns to step S86 in FIG. 8A. In this example, the predetermined time Tb is set to five minutes, for example, assuming a time required for the user to get up from a bed, use a bathroom, and return to the bed again from a clock time when the user presses down the measurement suspension switch 52C. However, the present disclosure is not limited to this.

As shown in step S86 in FIG. 8A, the CPU 110 determines whether it is a measurement clock time according to the schedule of the nocturnal blood pressure measurement mode. When it is a measurement clock time according to the schedule (Yes in step S86), the CPU 110 subsequently determines whether the measurement suspension state has been set. When the measurement suspension state has been set (Yes in step S87), the sphygmomanometer 100 cancels a blood pressure measurement process (step S88). In this example, as illustrated in FIG. 8D, the measurement set at 2:00 AM is canceled.

As shown in step S89 in FIG. 8A, the CPU 110 determines whether the given measurement set in the schedule of the nocturnal blood pressure measurement mode has been completed. When the given measurement has not been completed (incomplete in step S89), the processing returns to step S82.

As shown in step S82 in FIG. 8A, during standby, the CPU 110 determines whether the user has pressed down the measurement suspension switch 52C provided on the main body 10. The user is not required to press the measurement suspension switch 52C even after going to the bathroom. When the measurement suspension switch 52C has not been pressed down (No in step S82), the CPU 110 subsequently determines whether the measurement suspension timer of the sphygmomanometer 100 is on. When the measurement suspension timer is on (Yes in step S84), the sphygmomanometer 100 transitions to the measurement suspension timer process (step S85).

As shown in step S101 in FIG. 8C, in the measurement suspension timer process, the CPU 110 works as the recovery processing unit and causes the measurement suspension timer to count up. Subsequently, the CPU 110 determines whether the predetermined measurement post-suspension time Tb has elapsed (step S102). When the predetermined measurement suspension recovery time Tb has elapsed (Yes in step S102), the CPU 110 works as the recovery processing unit and resets the measurement suspension state (step S103). At this time, the indicator lamp 54 is turned on. Subsequently, the CPU 110 turns off the measurement suspension timer (step S104). Thereafter, the measurement suspension timer process is ended, and the processing returns to step S86 in FIG. 8A.

Note that, in step S82 in FIG. 8A, when the user has pressed the measurement suspension switch 52C after going to the bathroom (Yes in step S82), as shown in step S91 in FIG. 8B, in the measurement suspension switch process, the CPU 110 determines whether the sphygmomanometer 100 is in the measurement suspension state. When the sphygmomanometer 100 is in the measurement suspension state (Yes in step S91), the CPU 110 works as the recovery processing unit and resets the measurement suspension state (step S95). At this time, the indicator lamp 54 is turned on. Subsequently, the CPU 110 turns off the measurement suspension timer (step S96).

Thereafter, the measurement suspension switch process is ended, and the processing returns to step S84 in FIG. 8A.

As shown in step S86 in FIG. 8A, the CPU 110 determines whether it is a measurement clock time according to the schedule of the nocturnal blood pressure measurement mode. When it is a measurement clock time according to the schedule (Yes in step S86), the CPU 110 subsequently determines whether the measurement suspension state has been set. When the measurement suspension state has been reset (No in step S86), the sphygmomanometer 100 proceeds to the blood pressure measurement process (step S88). In this example, as illustrated in FIG. 8D, the measurement set at 3:30 AM in the schedule is performed.

As in the operation flow illustrated in FIG. 6C, the blood pressure measurement process shown in step S88 in FIG. 8A is performed according to steps similar to steps S2 to S11 excluding steps S5 and S6 in FIG. 5 described above. As shown in step S89 in FIG. 8A, the CPU 110 subsequently determines whether the measurement set in the schedule of the nocturnal blood pressure measurement mode has been completed. When all the given measurement has been completed (complete in step S89), the nocturnal blood pressure measurement mode of the sphygmomanometer 100 is ended. At this time, the indicator lamp 54 is turned off.

As is apparent from the above, according to the sphygmomanometer 100, it is possible to prevent the start of blood pressure measurement scheduled in advance in the nocturnal blood pressure measurement mode while the subject is temporarily out of bed.

In addition, the indicator lamp 54 is turned on while the sphygmomanometer 100 is in the nocturnal blood pressure measurement mode, and the indicator lamp 54 is temporarily turned off or blinking only while the sphygmomanometer 100 is in the measurement suspension state. Therefore, the user (subject) can check whether the sphygmomanometer 100 is in the nocturnal blood pressure measurement mode or in the measurement suspension state by viewing the indicator lamp 54.

### Modified Example

In each of the above-described examples, it is assumed that the nocturnal measurement switch 52B is pressed at 11:30 PM for transition to the nocturnal blood pressure measurement mode, and in the schedule of the nocturnal blood pressure measurement mode, for example, measurement is set at a fixed clock time of 2:00 AM and at 3:30 AM, that is 4 hours after a clock time when the nocturnal measurement switch 52B is pressed. However, the present disclosure is not limited to this schedule, and the schedule may be determined such that measurement is all set at fixed clock times such as 1:00 AM, 2:00 AM, and 3:00 AM between the clock time when the nocturnal measurement switch 52B is pressed and 7:00 AM, for example. Alternatively, the schedule may be determined such that measurement is all set at relative clock times such as, 2 hours after, 3 hours after, and 4 hours after the clock time when the nocturnal measurement switch 52B is pressed till 7:00 AM, for example.

In addition, since the sphygmomanometer 100 compresses a wrist (which is the left wrist 90 in the above examples, but may be a right wrist) as the measurement site, it is expected that the sphygmomanometer 100 hinders sleep of a user (subject) to a smaller extent than a sphygmomanometer that compresses an upper arm (Imai et al., “Development and evaluation of a home nocturnal blood pressure monitoring system using a wrist-cuff device”, Blood Pressure Monitoring 2018, 23, P318-326). Therefore, the sphygmomanometer 100 is suitable for nocturnal blood pressure measurement.

In addition, since the sphygmomanometer 100 is formed integrally and compactly as a wrist-type sphygmomanometer, it is handy for a user.

In the above embodiments, the sphygmomanometer 100 includes, as the operation unit 52, the measurement switch 52A, the nocturnal measurement switch 52B, and the measurement suspension switch 52C provided on the main body 10, but the present disclosure is not limited thereto. The operation unit 52 may include, for example, a communication unit that receives an instruction via wireless communication from a smartphone or the like outside of the sphygmomanometer 100.

In the above embodiments, the main body 10 is provided integrally with the cuff 20, but the present disclosure is not limited thereto. The main body 10 may be formed as a separate body from the cuff 20, and may be connected to the cuff 20 (fluid bag 22) via a flexible air tube for a fluid to be able to flow.

The above-described blood pressure measurement method may be recorded as software (computer program) on a recording medium capable of storing data in a non-transitory manner, such as a compact disc (CD), a digital versatile disc (DVD), or a flash memory. Installing the software recorded on such a recording medium in a substantial computer device such as a personal computer, a personal digital assistant (PDA), or a smartphone can cause the computer device to execute the above-described blood pressure measurement method.

As described above, a sphygmomanometer of the present disclosure that performs blood pressure measurement by temporarily compressing a measurement site of a subject with a blood pressure measurement cuff,

the sphygmomanometer having a nocturnal blood pressure measurement mode in which blood pressure measurement automatically starts according to a schedule determined in advance, the sphygmomanometer comprising:

a blood pressure measurement unit configured to automatically start blood pressure measurement according to the schedule and measure blood pressure when the blood pressure measurement cuff is in a pressurization process or a depressurization process, in the nocturnal blood pressure measurement mode;

a single operation switch for inputting an instruction to suspend the nocturnal blood pressure measurement mode or to recover to the nocturnal blood pressure measurement mode;

a suspension processing unit configured to perform a process for transitioning to a measurement suspension state in which blood pressure measurement does not start even when a clock time set in the schedule arrives, when the single operation switch is operated at a first time, in the nocturnal blood pressure measurement mode; and

a recovery processing unit configured to perform a process for recovering to the nocturnal blood pressure measurement mode, under condition that the single operation switch is operated at a second time or after a lapse of a predetermined time from a clock time when the single operation switch is operated at the first time, in the measurement suspension state.

Here, the “predetermined time” is set to five minutes, for example, assuming a time required for the subject to get up from a bed, use a bathroom, and return to the bed again. However, the present disclosure is not limited to this.

The sphygmomanometer of the present disclosure automatically starts blood pressure measurement according to the schedule in the nocturnal blood pressure measurement mode. The blood pressure measurement unit measures blood pressure when the blood pressure measurement cuff is in the pressurization process or the depressurization process. The instruction to suspend the nocturnal blood pressure measurement mode or to recover to the nocturnal blood pressure measurement mode is input to the single operation switch. The suspension processing unit performs the process for transitioning to the measurement suspension state in which blood pressure measurement does not start even when a clock time set in the schedule arrives, when the single operation switch is operated at the first time, in the nocturnal blood pressure measurement mode. The recovery processing unit performs the process for recovering to the nocturnal blood pressure measurement mode, under the condition that the single operation switch is operated at the second time or after a lapse of the predetermined time from the clock time when the single operation switch is operated at the first time, in the measurement suspension state. Therefore, according to the sphygmomanometer, it is possible to prevent the start of blood pressure measurement scheduled in advance in the nocturnal blood pressure measurement mode while the subject is temporarily out of bed.

In the sphygmomanometer of one embodiment, wherein the recovery processing unit is configured to, when complying with the condition that the single operation switch is operated at the second time, perform the process for recovering to the nocturnal blood pressure measurement mode immediately after the single operation switch is operated at the second time.

The sphygmomanometer of this embodiment can immediately recover to the nocturnal blood pressure measurement mode according to an instruction of the subject.

In the sphygmomanometer of one embodiment, wherein the recovery processing unit is configured to, when complying with the condition that the single operation switch is operated at the second time, perform the process for recovering to the nocturnal blood pressure measurement mode after a lapse of a predetermined time from a clock time when the single operation switch is operated at the second time.

Here, the “predetermined time” is set to five minutes, for example, assuming a time required for the subject to enter a resting state after pressing the single operation switch at the second time. However, the present disclosure is not limited to this.

In the sphygmomanometer of this embodiment, the process for recovering to the nocturnal blood pressure measurement mode is performed after a lapse of the predetermined time from the clock time when the single operation switch is operated at the second time. Therefore, it is possible to continue the nocturnal blood pressure measurement mode after waiting for the subject to enter the resting state.

In the sphygmomanometer of one embodiment, this sphygmomanometer comprises

an indicator lamp indicating whether the sphygmomanometer is in the nocturnal blood pressure measurement mode or in the measurement suspension state.

The sphygmomanometer of this embodiment allows the subject to check whether the sphygmomanometer is in the nocturnal blood pressure measurement mode or in the measurement suspension state by viewing the indicator lamp.

In the sphygmomanometer of one embodiment, wherein the measurement site is a wrist.

Since the sphygmomanometer of this embodiment compresses a wrist as the measurement site, it is expected that the sphygmomanometer hinders sleep of the subject to a smaller extent than a sphygmomanometer that compresses an upper arm (Imai et al., “Development and evaluation of a home nocturnal blood pressure monitoring system using a wrist-cuff device”, Blood Pressure Monitoring 2018, 23, P318-326). Therefore, this sphygmomanometer is suitable for nocturnal (sleep) blood pressure measurement.

In the sphygmomanometer of one embodiment, the sphygmomanometer comprises

a main body provided integrally with the blood pressure measurement cuff, wherein

the main body is equipped with the blood pressure measurement unit, the single operation switch, the suspension processing unit, and the recovery processing unit. Here, the “blood pressure measurement unit” includes, for example, a pump that supplies a pressurizing fluid to the blood pressure measurement cuff, a valve that releases the fluid from the blood pressure measurement cuff, and components that drive and control the pump, the valve, and the like.

The sphygmomanometer of this embodiment can be formed integrally and compactly. Therefore, the sphygmomanometer is handy for a user.

In another aspect, a blood pressure measurement method of the present disclosure for a sphygmomanometer that performs blood pressure measurement by temporarily compressing a measurement site of a subject with a blood pressure measurement cuff,

the sphygmomanometer

having a nocturnal blood pressure measurement mode in which blood pressure measurement automatically starts according to a schedule determined in advance, and

including a single operation switch for inputting an instruction to suspend the nocturnal blood pressure measurement mode or to recover to the nocturnal blood pressure measurement mode,

the blood pressure measurement method comprising:

automatically starting blood pressure measurement according to the schedule and measuring blood pressure when the blood pressure measurement cuff is in a pressurization process or a depressurization process, in the nocturnal blood pressure measurement mode;

performing a process for transitioning to a measurement suspension state in which blood pressure measurement does not start even when a clock time set in the schedule arrives, when the single operation switch is operated at a first time, in the nocturnal blood pressure measurement mode; and

performing a process for recovering to the nocturnal blood pressure measurement mode, under condition that the single operation switch is operated at a second time or after a lapse of a predetermined time from a clock time when the single operation switch is operated at the first time, in the measurement suspension state.

According to the blood pressure measurement method of the present disclosure, it is possible to prevent the start of blood pressure measurement scheduled in advance in the nocturnal blood pressure measurement mode while the subject is temporarily out of bed.

In yet another aspect, the computer-readable recording medium of the present disclosure is a computer-readable recording medium non-transitorily storing a program for causing a computer to execute the blood pressure measurement method.

The blood pressure measurement method can be implemented by making a computer read the program stored in the computer-readable recording medium of the present disclosure and causing a computer to execute the program.

As is clear from the above, according to the sphygmomanometer and the blood pressure measurement method of the present disclosure, it is possible to prevent the start of blood pressure measurement scheduled in advance in the nocturnal blood pressure measurement mode while the subject is temporarily out of bed. Furthermore, according to the program stored in the computer-readable recording medium of the present disclosure, it is possible to cause a computer to execute such a blood pressure measurement method.

The above embodiments are illustrative, and various modifications can be made without departing from the scope of the present invention. It is to be noted that the various embodiments described above can be appreciated individually within each embodiment, but the embodiments can be combined together. It is also to be noted that the various features in different embodiments can be appreciated individually by its own, but the features in different embodiments can be combined.

1. A sphygmomanometer that performs blood pressure measurement by temporarily compressing a measurement site of a subject with a blood pressure measurement cuff,
the sphygmomanometer having a nocturnal blood pressure measurement mode in which blood pressure measurement automatically starts according to a schedule determined in advance,
the sphygmomanometer comprising:
a blood pressure measurement unit configured to automatically start blood pressure measurement according to the schedule and measure blood pressure when the blood pressure measurement cuff is in a pressurization process or a depressurization process, in the nocturnal blood pressure measurement mode;
a single operation switch for inputting an instruction to suspend the nocturnal blood pressure measurement mode or to recover to the nocturnal blood pressure measurement mode;
a suspension processing unit configured to perform a process for transitioning to a measurement suspension state in which blood pressure measurement does not start even when a clock time set in the schedule arrives, when the single operation switch is operated at a first time, in the nocturnal blood pressure measurement mode; and
a recovery processing unit configured to perform a process for recovering to the nocturnal blood pressure measurement mode, under condition that the single operation switch is operated at a second time or after a lapse of a predetermined time from a clock time when the single operation switch is operated at the first time, in the measurement suspension state.

2. The sphygmomanometer according to claim 1, wherein the recovery processing unit is configured to, when complying with the condition that the single operation switch is operated at the second time, perform the process for recovering to the nocturnal blood pressure measurement mode immediately after the single operation switch is operated at the second time.

3. The sphygmomanometer according to claim 1, wherein the recovery processing unit is configured to, when complying with the condition that the single operation switch is operated at the second time, perform the process for recovering to the nocturnal blood pressure measurement mode after a lapse of a predetermined time from a clock time when the single operation switch is operated at the second time.

4. The sphygmomanometer according to claim 1, comprising
an indicator lamp indicating whether the sphygmomanometer is in the nocturnal blood pressure measurement mode or in the measurement suspension state.

5. The sphygmomanometer according to claim 1, wherein the measurement site is a wrist.

6. The sphygmomanometer according to claim 1, comprising
a main body provided integrally with the blood pressure measurement cuff, wherein
the main body is equipped with the blood pressure measurement unit, the single operation switch, the suspension processing unit, and the recovery processing unit.

7. A blood pressure measurement method for a sphygmomanometer that performs blood pressure measurement by temporarily compressing a measurement site of a subject with a blood pressure measurement cuff,
the sphygmomanometer
having a nocturnal blood pressure measurement mode in which blood pressure measurement automatically starts according to a schedule determined in advance, and
including a single operation switch for inputting an instruction to suspend the nocturnal blood pressure measurement mode or to recover to the nocturnal blood pressure measurement mode,
the blood pressure measurement method comprising:
automatically starting blood pressure measurement according to the schedule and measuring blood pressure when the blood pressure measurement cuff is in a pressurization process or a depressurization process, in the nocturnal blood pressure measurement mode;
performing a process for transitioning to a measurement suspension state in which blood pressure measurement does not start even when a clock time set in the schedule arrives, when the single operation switch is operated at a first time, in the nocturnal blood pressure measurement mode; and
performing a process for recovering to the nocturnal blood pressure measurement mode, under condition that the single operation switch is operated at a second time or after a lapse of a predetermined time from a clock time when the single operation switch is operated at the first time, in the measurement suspension state.

8. A computer-readable recording medium non-transitorily storing a program for causing a computer to execute the blood pressure measurement method according to claim 7.
